(19) United States
(12) Patent Application Publication
(10) Pub. No.: US 2024/0407802 A1
(43) Pub. Date: Dec. 12, 2024

(54) ASPIRATION DEVICES FOR TREATMENT OF THROMBOSIS INCLUDING EXPANDABLE DISTAL ENDS AND SYSTEMS AND METHODS THEREOF

(71) Applicant: Caeli Vascular, Inc., St. Louis, MO (US)

(72) Inventors: Mohamed ZAYED, Chesterfield, MO (US); Guy GENIN, St. Louis, MO (US); Usama ISMAIL, Clayton, MO (US); Roger ROWE, St. Louis, MO (US)

(21) Appl. No.: 18/603,717

(22) Filed: Mar. 13, 2024

Related U.S. Application Data
(63) Continuation of application No. PCT/US2022/076597, filed on Sep. 16, 2022.
(60) Provisional application No. 63/245,696, filed on Sep. 17, 2021.

(51) Int. Cl. A61B 17/3207; A61B 17/00

(52) U.S. Cl. CPC A61B 17/320758; A61B 2017/00778; A61B 2017/00871; A61B 2017/320775; A61B 2217/005; A61B 2217/007

(57) ABSTRACT

Described herein are systems, devices, and methods for treating an intravascular thrombosis. In some embodiments, an apparatus may include a catheter and an agitation device slidably coupled to the catheter, the agitation device comprising at least one agitation element configured to reshape or macerate a thrombus.

### CROSS-REFERENCE TO RELATED APPLICATIONS

This application claims priority to U.S. Provisional Application No. 63/245,696, filed Sep. 17, 2021, the entire disclosure of which is incorporated herein by reference in its entirety.

### TECHNICAL FIELD

The embodiments described herein relate generally to systems, devices, and methods for treatment of thrombosis, and more specifically, to a catheter system including one or more sets of agitation elements.

### BACKGROUND

Deep vein thrombosis (DVT) is a potentially deadly medical condition that is costly to treat and that impacts hundreds of thousands of Americans every year. DVT is characterized by the formation of blood clots within the deep venous system of the body that, if they fracture or dislodge either spontaneously or during surgery or treatment, can lead to pulmonary embolism (PE), which can in turn lead to death or significant morbidity. Current treatment options for DVT, PE, or other types of thrombosis are limited in both effectiveness and safety for patients, in part because the difficulty in removing large thrombi from vessels. In some cases, thrombolytic drugs may be used, but the effects of such drugs when not confined to a defined region may produce undesirable results such as bleeding. Debris from a thrombectomy or other clot removal procedure may also create complications, e.g., when such debris become lodged in other anatomical regions.

Intravascular agitators can be used to dissociate, breakdown, or fragment larger intravascular material such as thrombi to facilitate their removal from a vessel. Agitator efficiency is dependent on its ability to reach the full circumference of the lumen of the vessel, while accounting for possible intraluminal wall irregularly, vessel curvature, vessel tortuosity, and mechanical properties of the intraluminal vessel contents. Since agitators can also potentially impact the vessel wall including the endothelium, it is imperative that the agitator is an effective fragmentation tool while also minimizing the risk of damage to the vessel wall.

### SUMMARY

Described herein are systems, devices, and methods for treating thrombosis. Intravascular agitators are used to dissociate, breakdown, or fragment unwanted intravascular material into smaller portions. Agitator efficiency is dependent on its ability to reach the full circumference of the lumen of the vessel, while accounting for possible intraluminal wall irregularity, vessel curvature, vessel tortuosity, and mechanical properties of the intraluminal vessel contents. Since agitators can also potentially impact the vessel wall including the endothelium, it is imperative that the agitator is an effective fragmentation tool while also minimizing the risk of damage to the vessel wall.

Embodiments and examples disclosed herein generally relate to a intraluminal agitator that is inserted over and/or through a vascular catheter for the purpose of disrupting and/or fragmenting blood clots. The agitator allows for advancement, retraction, and movement within a defined treatment region to dislodge and fragment blood clots. The agitator allows for circumferential rotation and can rotate 360 degrees about the longitudinal axis of the vasculature. The agitator includes multiple open loops, each of which may contribute to the fragmentation of blood clots. The agitator is atraumatic in design. In some embodiments, the agitator can include nitinol wire loops that will cause minimal vein wall trauma during use by optimizing the gauge of wire used as well as the shape of the loops. The agitator accommodates various vessel lumen dimensions and is passively compressible and expandable allowing for usage in a range of vascular diameters by having agitator loops with flexural rigidity that both exert shear forces on blood clots and limit forces on the vessel wall. The agitator is configured such that, in cases of a defined treatment zone, portions of the agitator wires may be directed to contact, displace, and/or fragment thrombus in the extremities of the treatment zone. The agitator is configured to protect the extremities of the treatment zone from being compromised, displaced, or broken during movement of the agitator. The agitator may pass over an inner catheter and is configured to allow for thrombolytic drugs or other fluids to be infused into the treatment zone. Alternatively, thrombolytic drugs or other fluids may be infused directly through the lumen of the agitator into the treatment zone. The agitator loops can be angled to engage thrombus or thrombus fragments in difficult to reach regions within a vessel.

In some embodiments, an apparatus may include a catheter and an agitation device slidably coupled to the catheter, the agitation device including at least one agitation element configured to macerate a thrombus.

In some embodiments, a catheter system includes a guidewire, a catheter configured to follow the guidewire, and an agitation device slidably coupled to the catheter, the agitation device comprising at least one agitation element configured to macerate a thrombus.

In some embodiments, a catheter system includes a method of removing a thrombus from a blood vessel. The method includes advancing a catheter system to a target site containing a thrombus, deploying an agitation device of the catheter system, macerating the thrombus via the agitation device, removing the thrombus fragments, and removing the catheter system.

### BRIEF DESCRIPTION OF THE DRAWINGS

FIG. 1 is a schematic diagram of a catheter system for treating thrombosis, according to embodiments.

FIG. 2 is a schematic diagram of a distal portion of a catheter system for treating thrombosis, according to embodiments.

FIG. 3 is a schematic diagram of an agitation device for treating thrombosis, according to embodiments.

FIG. 4 is a perspective view of an agitation device for treating thrombosis, according to embodiments.

FIG. 5A is a side view of the agitation device of FIG. 4.

FIG. 5B is a front view of the agitation device of FIG. 4.

FIG. 6 is a perspective view of a catheter system including an agitation device for treating thrombosis, according to embodiments.

FIG. 7 schematically depicts a region in which an agitation element of an agitation device can be orientated, according to embodiments.

FIGS. 8A-8C schematically depict different fenestration patterns of an agitation device for treating thrombosis, according to embodiments.

FIG. 9 is a flow diagram of a method of performing a thrombectomy procedure, according to embodiments.

### DETAILED DESCRIPTION

Described herein are systems, devices, and methods for removing material (e.g., a blood clot or thrombus) from a vessel of a subject. Systems, devices, and methods described herein include agitation devices for macerating a thrombus to facilitate thrombus removal.

The agitation devices or agitators described herein can be used to aid the removal of thrombus in blood vessels of patients. The agitators can be designed to treat a variety of different vessel diameters and sizes in a safe and efficient manner. In particular, the agitators can allow a physician to dissociate, pull back, and disrupt thrombus laden in a treatment region.

In some implementations, the agitator is constructed by wrapping and bonding of nitinol (or other suitable metals or plastics) wires to the outer surface of a thin-walled cannula. In some implementations, all loops are constructed through the bonding of a single strand or multiple, continuous, strands of wire to the cannula. In other implementations, subsets of the loops are constructed through the bonding of a single strands or multiple, continuous, strands of wire to the cannula.

Systems, devices, and methods described herein can be configured to define a sequestered treatment zone or treatment area in which thrombus can be fragmented through the action of an agitation device or agitator. The agitator serves dual roles: dislodging thrombus from a vessel wall and fragmenting or reshaping thrombus into pieces sufficiently small to be evacuated from the treatment region, e.g., via suction.

Examples of catheter systems including agitators for removing thrombus are described in U.S. Patent Application Publication No. 2022/0047282, filed Sep. 26, 2019, and titled “Balloon Encapsulation and isovolumetric suction Thrombectomy Catheter and Methods Thereof,” the disclosure of which is hereby incorporated by reference in its entirety.

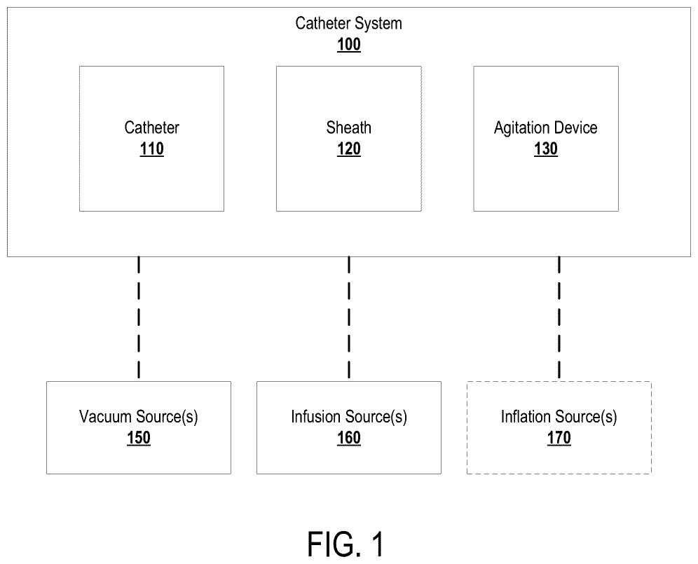

FIG. 1 schematically depicts a catheter system 100 for treating thrombosis, according to embodiments. The catheter system 100 can include a catheter 110, a sheath 120, and an agitation device 130. One or more of the catheter 110, the sheath 120, or the agitation device 130 can include one or more ports or connectors, e.g., for connecting to one or more of a vacuum source 150, an infusion source 160, and an inflation source 170. For example, one or more of the catheter 110, the sheath 120, or the agitation device 130 can include a manifold or other structure with ports configured to connect to one or more of a vacuum source 150, an infusion source 160, and an inflation source 170.

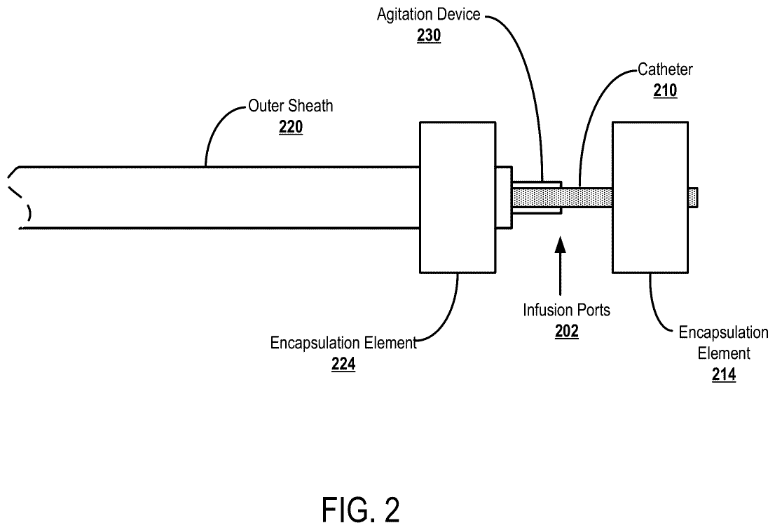

The catheter 110 can include a proximal portion, an elongate body, and a distal portion. The proximal portion of the catheter 110 can include one or more ports or connectors for connecting to a vacuum source 150, an infusion source 160, and an inflation source 170. The distal portion of the catheter 110 can include one or more infusion ports, e.g., for delivering fluid into a treatment region. The one or more infusion ports can be connected to an infusion source (e.g., infusion source 160) via a lumen defined by the elongate body of the catheter 110. The distal portion of the catheter 110 can also include one or more occlusion elements or encapsulation elements, as further described with reference to FIG. 2. In some embodiments, the one or more encapsulation elements can be expandable or inflatable balloons, which can be deployed (e.g., expanded or inflated) by delivering a fluid (e.g., gas or liquid) from an inflation source (e.g., inflation source 170) to the encapsulation element. The encapsulation element can be coupled to the inflation source via a lumen defined by the elongate body of the catheter 110. In some embodiments, the distal portion of the catheter 110 can also include an introducer tip or atraumatic tip, e.g., for insertion and advancement of the catheter 110 (with other portions of the catheter assembly 100) into a vessel of a patient, e.g., to a treatment site including a thrombus. In some embodiments, the elongate body of the catheter (with other portions of the catheter assembly 100) 110 can be configured to define a lumen for receiving a guidewire, e.g., for tracking of the catheter along a guidewire.

The sheath 120 can include a proximal portion, an elongate body, and a distal portion. The elongate body of the sheath 120 can be configured to define a channel or lumen for receiving the elongate body of the catheter 110. In some embodiments, the catheter 110 can be slidably disposed within the lumen of the sheath 120. The proximal portion of the sheath 110 can include one or more ports or connectors for connecting to a vacuum source 150, an infusion source 160, and an inflation source 170. The distal portion of the sheath 120 can include one or more occlusion elements or encapsulation elements, as further described with reference to FIG. 2. In some embodiments, the one or more encapsulation elements can be expandable or inflatable balloons, which can be deployed (e.g., expanded or inflated) by delivering a fluid (e.g., gas or liquid) from an inflation source (e.g., inflation source 170) to the encapsulation element. The encapsulation element can be coupled to the inflation source via a lumen defined by the elongate body of the sheath 120. In some embodiments, the catheter 110 and the sheath 120 can slide or move relative to one another, e.g., to change a distance between the encapsulation elements respectively disposed thereon. When deployed, the encapsulation elements on the catheter 110 and the sheath 120 can collectively define and/or isolate a treatment area around a thrombus, e.g., for delivering thrombolytic treatment, fragmenting or macerating clot and removing clot particles and/or fluids from the vessel. Details of such a treatment area are described in further detail with respect to FIG. 2. In some embodiments, the sheath 120 can include or define a lumen that can be coupled to a vacuum source (e.g., vacuum source 150). The vacuum source can be configured to apply suction via the lumen of the sheath 120, e.g., to remove fluid and/or particles of thrombus from a vessel or treatment area. Alternatively, in some embodiments, the vacuum source can be configured to apply suction via a lumen of the catheter 110 instead of the sheath 120. In such embodiments, the sheath 120 can include one or more infusion ports or openings, and the lumen of the sheath 120 via the infusion openings can be configured to deliver fluids (e.g., thrombolytic agents, therapeutic agents, saline, etc.) into the treatment area.

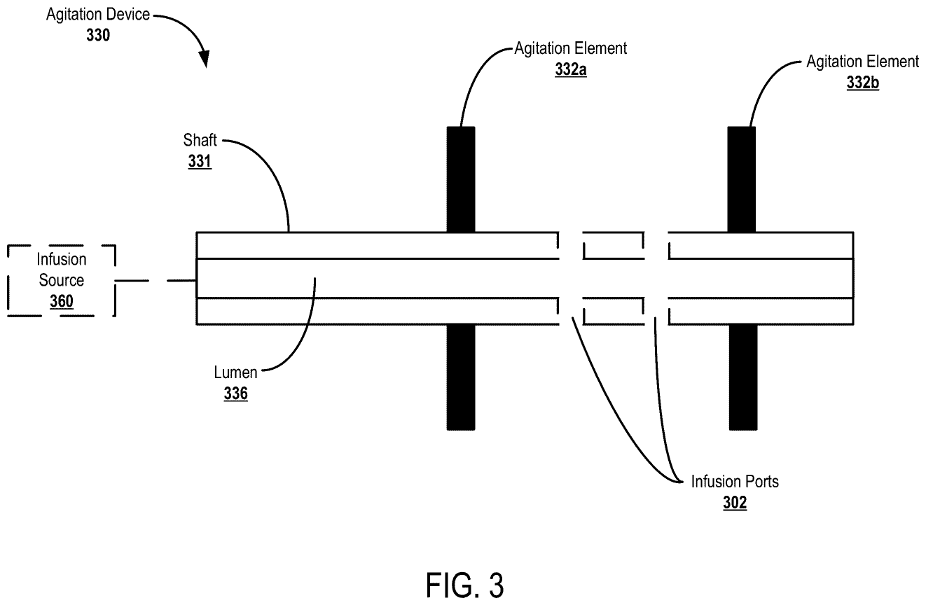

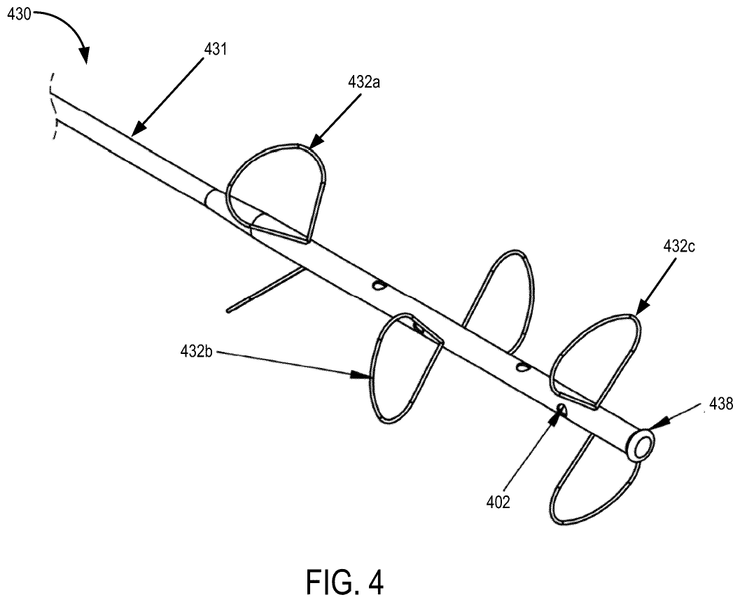

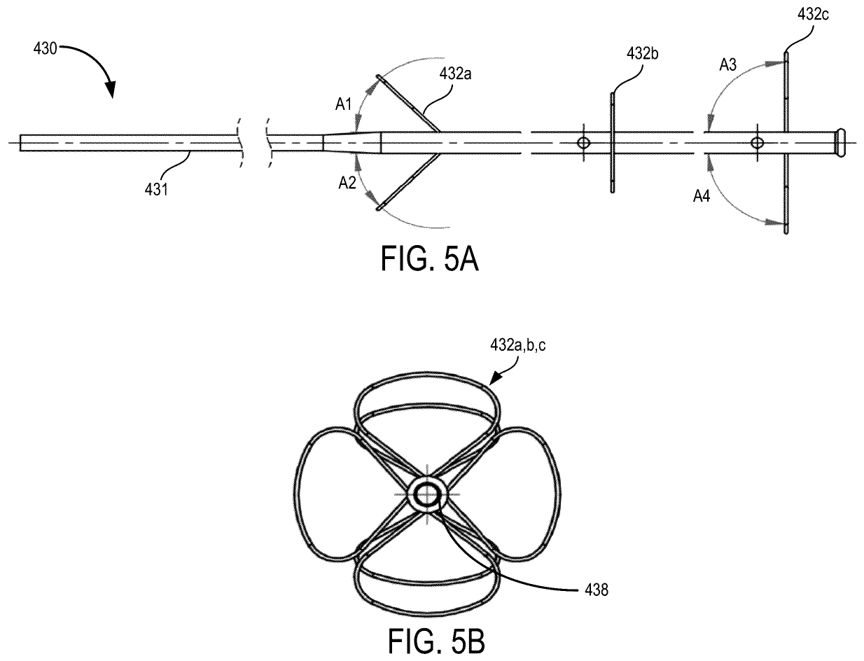

The agitation device 130 can be configured to fragment, macerate, morcellate, or otherwise break up a clot or thrombus. In some embodiments, the agitation device 130 can include one or more sets of agitation elements. The agitator device with the agitation elements can be configured to move (e.g., translate and/or rotate) to mechanically break apart a larger thrombus into smaller pieces to allow it to be either suctioned through a suction lumen (e.g., defined by the sheath 120, as described above) or contained between proximal and distal encapsulation elements as the catheter system is removed from the patient. The agitation elements can be configured to extend outward from a shaft of the agitation device 130, e.g., in a direction toward the vessel wall. In some embodiments, the agitation elements can be deformable or flexible, such that the agitation elements can deform to fit within a vessel based on its shape. In some embodiments, the agitation elements are configured to extend a full length from a shaft of the agitation device 130 to the vessel wall, such that the agitation elements can be configured to break up clot within that entire region. Further details of the agitation elements and other components of an agitation device 130 are described with reference to FIG. 3. The agitator accommodates various vessel lumen dimensions and is passively compressible and expandable allowing for usage in a range of vascular diameters by having agitation elements with flexural rigidity that both exert shear forces on blood clots and limit forces on the vessel wall. In some embodiments, the geometry or flexural rigidity of the agitation elements is tapered spatially so as to optimize the removal of the apparatus from the treatment zone via a catheter lumen, as depicted in FIGS. 4-5B.

The vacuum source 150 can coupled to the catheter system 100 to apply suction, e.g., to remove fluid and/or particles from a vessel or treatment area. The vacuum source 150 may be a rotary vane pump, a diaphragm pump, a liquid ring, a piston pump, a scroll pump, a screw pump, a roller pump, a peristaltic pump, a syringe with a plunger or another pump configured to draw fluid and/or particles from the vessel or treatment area. The vacuum source 150 is configured to provide vacuum pressure capable of removing fluid and/or particles without damaging the vessel. In some embodiments, the vacuum source 150 may be coupled to a container configured to capture fluid and/or particles. Examples of suitable vacuum pumps are described in PCT Application Publication No. WO 2021/155293, filed Jan. 29, 2021, and titled “Isovolumetric pump and systems and methods thereof,” the disclosure of which is hereby incorporated by reference in its entirety.

The infusion source 160 can be fluidically coupled to the catheter system 100 to deliver fluid to the treatment area. For example, the infusion source 160 may be configured to deliver one or more of a therapeutic agent, a numbing agent, a dilating agent, a thrombolytic agent, a diagnostic agent such as vascular contrast solution or the like to affect the vessel at the treatment area. In some embodiments, the infusion source 160 may be a reservoir, a syringe, or other fluid-containing device. The infusion source 160 may deliver fluid to the treatment area for a predetermined period, may deliver fluid intermittently, or may be manually controlled to deliver fluid only when activated (e.g., via an actuator) by a medical professional. In some embodiments, the infusion source 160 may include or be coupled to a pump or similar device to aid in delivering the fluid into the vessel or treatment area. In some embodiments, the infusion source 160 may be a manually actuated device (e.g., a syringe) for manually infusing fluid into the vessel.

The inflation source 170 can be fluidically coupled to the catheter system 100 for inflating one or more encapsulation elements with an inflation fluid (e.g., liquid, gas, etc.). The inflation source 170 may be or include a pump or similar device for generating fluid flow into the encapsulation elements to inflate them. In some embodiments, the catheter system 100 can include components (e.g., sensors) for preventing overinflation of the encapsulation elements. For example, the inflation source 170 may include a gauge that measures the pressure or volume within the encapsulation elements. In some embodiments, the inflation source 170 may inflate the encapsulation elements to a predetermined pressure or volume. The inflation source 170 may be further configured to apply a vacuum to deflate or partially deflate the encapsulation elements.

In some embodiments, the catheter system 100 may not include an inflation source. In such embodiments, the catheter system 100 can have encapsulation elements that can be expanded without using an inflation source. For example, in some embodiments, the encapsulation elements can be self-expandable. The encapsulation elements can expand when a sheath or other structure compressing the encapsulation elements is withdrawn, or when the encapsulation elements are extended out of the sheath. In some embodiments, the encapsulation elements can include manually actuatable encapsulation elements, such as, for example, deployable baskets, meshes, screens, filters etc. For example, a first end of the encapsulation element can be coupled to a first shaft, while a second end of the encapsulation element can be coupled to a second shaft, and relative movement of the shafts relative to one another can cause the encapsulation elements to expand or retract/compress.

FIG. 2 schematically depicts a distal portion of a catheter system for treating thrombosis, according to embodiments. The catheter system depicted in FIG. 2 can be structurally and/or functionally similar to that depicted in FIG. 1, and therefore can include similar components as those depicted in FIG. 1. For example, the catheter system can include a catheter 210 (e.g., functionally and/or structurally similar to the catheter 110) including infusion ports 202, a first encapsulation element 214, a second encapsulation element 224, an agitation device 230 (e.g., functionally and/or structurally similar to the agitation device 130), and an outer sheath 220 (e.g., functionally and/or structurally similar to the sheath 120).

The catheter 210 is disposed within the lumen of the outer sheath 220 and is configured to slide relative to the outer sheath 220. The catheter 210 can be coupled to an infusion source (e.g., infusion source 160) for supplying a fluid into a treatment area. The catheter 210 can include at its distal end one or more infusion ports 202, which are configured to deliver the fluid into the treatment area from the infusion source. The infusion ports 202 may have any shape suitable for delivering fluid into the treatment area. In some embodiments, the infusion ports 202 may cover or extend along an entire length of the catheter disposed between the first and second encapsulation elements 214, 224. Alternatively, the infusion portions may be disposed in select regions of the catheter between the first and second encapsulation elements 214, 224. In some embodiments, the position of the catheter 210 can be adjusted relative to the outer sheath 220, such that a selective number or length of infusion portions 202 is exposed for delivering a fluid into the treatment area. This can allow a user to adjust the rate of infusion delivery. In some embodiments, the infusion ports 202 can be disposed around an entire circumference of the catheter. Alternatively, the infusion ports 202 can be disposed around a portion of the circumference of the catheter, e.g., up to about 270 degrees, up to about 180 degrees, or up to about 90 degrees, including all sub-ranges and values therebetween. In some embodiments, the infusion ports 202 can be directionally placed (e.g., along one side of the catheter) such that the infusion ports 202 can directionally deliver fluid into the treatment area. In some embodiments, the catheter 210 can be rotatable such that the direction of the infusion ports 202 can be adjusted.

While the infusion ports 202 are described as being disposed on the catheter 210, it can be appreciated that the infusion ports 202 can be disposed on other components of the catheter system, including, for example, the agitation device 230, the outer sheath 220, or one or both of the encapsulation elements 214 and 224.

The first encapsulation element 214 and the second encapsulation element 224 are configured to be deployed (e.g., inflated or expanded) or undeployed (e.g., deflated or retracted), e.g., by one or more inflation sources. In some embodiments, the first and second encapsulation elements 214 and 224 can be expandable balloons. Alternatively, the first and second encapsulation elements 214 and 224 can be expandable mesh structures, baskets, cages, screens, filters, or the like. The first encapsulation element 214 can be coupled to the distal portion of the catheter 210, and the second encapsulation element 224 can be coupled to the distal portion of the outer sheath 220. The area between the first encapsulation element 214 and the second encapsulation element 224 can define an isolated space or isolated treatment area when the encapsulation elements 214 and 224 are deployed in a vessel. In particular, isolation occurs when the first encapsulation element 214 and the second encapsulation element 224 are deployed (e.g., inflated, filled, etc.) in a vessel and contact the vessel walls, thereby forming a seal with the vessel wall and preventing fluid, thrombus, and/or thrombus particles from leaving the treatment area. In some embodiments, the length of the treatment area (e.g., distance between the first encapsulation element 213 and the second encapsulation element 224) is fixed. In some embodiments, the length may be adjusted by moving (e.g., sliding) the catheter and the outer sheath 220 relative to one another. For example, if a thrombus or clot were disposed along a longer length of vessel, the catheter 210 (and the first encapsulation element 214) may be disposed farther from the outer sheath 220 (and the second encapsulation element 224) to define a larger treatment area. As such, the exact location and the size of the treatment area may be determined by the size and location of the thrombus. When the encapsulation elements 214 and 224 are expanded, they can isolate the thrombus for treatment, e.g., breaking, macerating, and removal.

While not depicted, it can be appreciated that the outer sheath 220 can include one or more lumens for supplying fluid (e.g., liquid or gas) to the encapsulation element 224, e.g., to expand or deploy it. Similarly, the catheter 210 can include one or more lumens for supplying fluid (e.g., liquid or gas) to the encapsulation element 214, e.g., to expand or deploy it. In some embodiments, the encapsulation elements 214 and 224 can each include multiple expandable portions that are selectively and/or independently expandable. For example, each expandable element 214 and 224 can be formed of a plurality of expandable balloons that can be selectively and independently expanded.

The agitation device 230 can include one or more agitation elements that are configured to engage with and reshape, macerate, or break apart a thrombus or clot. In some embodiments, the agitation device 230 can include a shaft that is concentrically arranged with respect to the outer sheath 220 and the catheter 210. The shaft can be configured to slide or move relative to the outer sheath 220 and the catheter 210. In some embodiments, the shaft can be disposed between the outer sheath 220 and the catheter 210. In use, the shaft of the agitation device 230 can be extended beyond a distal end of the outer sheath 220 and into the treatment area. The agitation elements of the agitation device 230 can then be used to engage with the clot to reshape, macerate, or break apart the clot for removal from the treatment area. In some embodiments, the agitation elements of the agitation device 230 can be configured to transition from an undeployed state to a deployed state. For example, the agitation elements can be held in a compressed or undeployed state prior to exiting the outer sheath 220, and then self-expand or self-deploy into an expanded or deployed state. In such embodiments, the agitation elements can be formed of memory set materials, e.g., nitinol. In some embodiments, a surgeon or other medical professional can actuate a button, knob, or other actuation device to deploy the agitation elements.

In some embodiments, the agitation elements of the agitation device 230 can be manipulated or moved (e.g., rotated, translated, vibrated, oscillated, etc.) within the treatment area, e.g., to aid in reshaping and/or breaking apart the thrombus. The agitation elements can include loops, spikes, or other mechanical structures suitable for breaking apart the thrombus. The agitation elements, by being movable within the treatment area, can be configured to engage with thrombus or clot throughout the entire length of the treatment region (or a substantial portion of the length of the treatment region) to facilitate removal of the thrombus. In some embodiments, the agitation elements, when expanded or deployed, can be configured to contact a wall of the vessel. As such, the agitation elements can extend to cover a full radial or lateral dimension of the treatment region, as defined by the wall of the vessel. Depending on the location of the treatment area within the vasculature, the vessel diameter may vary. Therefore, the agitation elements can be configured to extend to fit within a range of vessel diameters. The agitation elements can be compliant and/or flexible, such that they can extend to contact or engage the vessel wall but be sufficiently flexible so as to not damage or injure the vessel. The agitation elements, when moved (e.g., rotated, translated, etc.) within the vessel, can then engage clot along the full radial or lateral dimension of the vessel. This can increase the effectiveness of the agitation elements at removing thrombus from the treatment area.

In some embodiments, the distal portion of the catheter system, including the catheter 210, the outer sheath 220, and the agitation device 230, can be sufficiently flexible to allow access into peripheral vasculature, e.g., via an internal jugular vein, femoral vein, or femoral artery.

While not depicted, in some embodiments, the catheter 210 and/or the sheath 220 can also include agitation elements, e.g., loops, wires, baskets, and/or other mechanical structure suitable for reshaping, breaking and/or fragmenting thrombus.

FIG. 3 schematically depicts an agitation device 330 (e.g., functionally and/or structurally similar to the agitation device 130 and/or 230) for treating thrombosis, according to embodiments. The agitation device 330 can include a shaft 331 with agitation elements 332a and 332b extending radially away from the shaft 331. The agitation elements 332a and 332b can be mechanical structures that are configured to reshape, fragment, or break apart a clot. The agitation elements 332a and 332b may be disposed anywhere along the shaft 331 and may be configured and/or arranged suitably for thrombus engagement. The agitation elements 332a and 332b can be configured to remove thrombus from a vessel wall and to fragment the thrombus into small enough pieces to be evacuated from the treatment area, e.g., via suction (such as through a catheter such as outer sheath 120 or 220).

In some embodiments, the agitation device 330 can include any number (e.g., about 1, 2, 3, 4, 5, 6, 7, 8, 9, 10, 11, 15, 20, 25, 30, 40, 50, 100, etc.) of agitation elements. In some embodiments, the agitation device 330 may include multiple sets of agitation elements 332a, 332b. For example, a first set of agitation elements can be disposed at a first location along the shaft 331, and a second set of agitation elements can be disposed at a second location along the shaft 331. The first and second locations can be spaced from one another by a predetermined distance. Each set of agitation elements can include one or more agitation elements. In some embodiments, the agitation device 330 can include between 1 and 6 sets of agitation elements.

During use, the shaft 331 can be rotated and/or translated, to cause the agitation elements 332a and 332b to move (e.g., rotate and/or translate). In some embodiments, the agitation elements 332a and 332b may be configured to transition from an undeployed state to a deployed state. For example, the agitation elements 332a and 332b may be configured to be in an undeployed state (e.g., compressed state) during delivery through the vasculature to a treatment area, and to transition to a deployed state (e.g., expanded state) once in the treatment area. In some embodiments, the agitation elements 332a and 332b can be formed from a memory set material, such that the agitation elements 332a and 332b can self-expand within the treatment area. The agitation elements 332a and 332b may be self-expanding and may expand to a pre-set shape and diameter when unconstrained or released form an outer constraint, e.g., by advancing the shaft 331 beyond the lumen of a sheath (e.g., sheath 120 or 220). The agitation elements 332a and 332b can also be collapsed to a smaller diameter, e.g., by moving the shaft 331 inside the lumen of a sheath (e.g., sheath 120 or 220). Alternatively, the agitation elements 332a and 332b can be configured to expand in response to an actuation by a user. The agitation elements 332a and 332b can also be passively compressible and expandable allowing for usage in a range of vascular diameters and allowing for the agitation elements 332a and 332b to exert forces on clots within and up to the vessel wall while limiting forces on the wall.

The agitation elements 332a and 332b can be flexible but have sufficient strength to reshape, fragment, or break up a clot. In some embodiments, the agitation elements 332a and 332b can be used with a thrombolytic or softening agent that is introduced first into the treatment area, e.g., to soften, break apart, or dissociate the thrombus. In some embodiments, the agitation elements 332a and 332b, when expanded without any restriction, can be configured to have a diameter that is equal to or larger than an inner diameter of a vessel being treated. As such, when the agitation elements 332a and 332b are deployed within the treatment area within the vessel, the agitation elements 332a and 332 can be configured to expand to contact the walls of the vessel. In some embodiments, where the agitation elements 332a and 332b have a diameter larger than the vessel, the agitation elements 332a and 332b can be configured to contact and then deform to the size and/or shape of the vessel.

The shaft 331 may be sufficiently flexible for navigation through a patient's vasculature, including a patient's peripheral vasculature. The agitation device 330, like agitation devices 130 and 230, can be configured to track along a catheter (e.g., catheter 110 or 210) and/or within an outer sheath (e.g., outer sheath 120 or 220) to a treatment area. In some embodiments, the shaft 331 may be of consistent outer diameter (or dimension if non-circular) along its length of may have one or more changes in outer diameter (or dimension if non-circular) along its length. For example, in some embodiments, the shaft 331 may be configured to taper in size or have stepped decreases in size going from its proximal end to its distal end.

The agitation elements 332a and 332b are of an atraumatic design, e.g., are configured to have atraumatic ends. The atraumatic ends of the agitation elements 332a and 33b can be configured to reduce trauma or injury to vessel walls during use. In some embodiments, the agitation elements 332a and 332b may be formed of a single strand or multiple strands of wire or fiber. The agitation elements 332a and 332b may be made from a biocompatible material such as a polymer or a metal. In some embodiments, the agitation elements 332a and 332b are formed of at least one of polyether ether ketone or PEEK, Pebax, or nitinol alloy. In some embodiments, the agitation elements 332a and 332b are formed of a single material (e.g., nitinol). In some embodiments, the agitation elements 332a and 332b may be formed of multiple biocompatible materials in layered, braided, wrapped, or gradated configurations. When the agitation elements 332a and 332b are formed of one or more wires, the gauge of wire, or diameter of material, used to form the agitation elements 332a and 332b can be selected to reduce vessel wall trauma.

In some embodiments, different agitation elements 332a and 332b can have different structure, shape, size, etc. For example, different gauges of wire can be used to form different agitation elements 332a and 332b. In some embodiments, the first agitation element 332a, when deployed or expanded, can have a larger outer diameter or dimension than the second agitation element 332b. In some embodiments, the first and second agitation elements 332a and 332b can be set at different angles relative to a longitudinal axis of the shaft 331. In some embodiments, the first and second agitation elements 332a and 332b can be formed of different materials, e.g., one being formed of nitinol while another being formed of fibers or a polymer. By having the first and second agitation elements 332a and 332b be differently configured, they can be used for different purposes, e.g., one for reshaping the thrombus or drawing it in, and another for breaking or fragmenting a thrombus. The different configurations of the first and second agitation elements 332a and 332b can be selected to optimize thrombus removal.

In some embodiments, the agitation device 330 may be optionally coupled to an infusion source 360 (e.g., functionally and/or structurally like the infusion source 160) by a lumen 336 and one or more connection ports. In some embodiments, the extracorporeal portion of the agitator shaft can terminate with a hemostatic valve and an access port (e.g., a Luer-terminated port), allowing for liquids to be introduced within the agitator shaft and transported to the treatment area. The lumen 336 can be configured to convey fluid from the infusion source 360 to one or more infusion ports disposed in the treatment area, e.g., near the agitation elements 332a and 332b. The infusion ports 302 can be configured to deliver one or more therapeutic agents, thrombolytic agents, contrast agents, or like fluids into the treatment region. Alternatively, the agitation device 330 may not be coupled to an infusion source 360. In such cases, one or more other components of the catheter system can be configured to deliver infusion fluid, e.g., such as the catheter and/or outer sheath. The extracorporeal portion of the agitator shaft may be terminated with a hemostatic valve allowing for hemostasis to be maintained with another vascular device advanced through the shaft's inner lumen.

FIG. 4 depicts a perspective view of an agitation device 430 (e.g., functionally and/or structurally similar to the agitation device 130, 230, or 330) for treating thrombosis, according to embodiments. The agitation device 430 includes a shaft 431 (e.g., functionally and/or structurally similar to the shaft 331) with a plurality of infusion ports (e.g., functionally and/or structurally similar to the infusion ports 302), and agitation elements (e.g., functionally and/or structurally similar to the agitation elements 332a, 332b) including a first set of agitation elements 432a, a second set of agitation elements 432b, and a third set of agitation elements 432c. Given the similarities between the agitation device 430 and other agitation devices and components described herein, certain details of such components are not described herein again.

The shaft 431 extends to a distal end that may or may not include a stopper or tip 438. If present, the stopper 438 has an atraumatic structure, which can be configured to prevent the agitation device 430 from dislodging an encapsulation element (e.g., encapsulation elements 214 or 224) or other elements that define an isolated treatment area. As such, the stopper 438 can be configured to prevent compromise of the isolated treatment area.

In the embodiment depicted in FIG. 4, the sets of agitation elements 432a, 432b, and 432c includes loops having a clover-leaf shape. The loops can be attached at the shaft 431 at one end and configured to engage with a vessel wall at the other end. Each set of agitation elements 432a, 432b, and 432c can include two loops, which can be spaced from each other by an angle of approximately 180 degrees. While two loops are shown in each set 432a, 432b, and 432c, with the loops evenly spaced about the circumference of the shaft 431, it can be appreciated that any number of loops can be in each set 432a, 432b, and 432c, and that the loops can be evenly spaced or spaced at other degrees relative to one another. For example, the agitation device 430 can include between 1 and about 25 agitation elements per set, including all values and sub-ranges therebetween. any number of agitation elements. In some embodiments, each set of agitation elements 432a, 432b, and 432c may include uniform (e.g., the same) agitation elements or may include agitation elements with varying characteristics (e.g., shape, height, width, angle, material, etc.).

The sets of agitation elements 432a, 432b, and 432c can be arranged with the loops of adjacent sets being offset from one another. For example, the loops of the second set 432b can be offset 90 degrees about the circumference of the shaft 431 relative to the loops of the first set 432a. Similarly, the loops of the third set 432c can be offset 90 degrees about the circumference of the shaft 431 relative to the loops of the second set 432b. Additionally or alternatively, the sets of agitation elements 432a, 432b, and 432c can be angled relative to the longitudinal axis of the shaft 431 at different angles, as better depicted in the side view of the agitation device 430, shown in FIG. 5A.

As shown in FIG. 5A, the first set of agitation elements 432a is configured with a first loop forming an angle A1 with the shaft 43 land a second loop forming an angle A2 with the shaft 431. In some embodiments, the angle A1 and the angle A2 can be the same, while in other embodiments, the angle A2 can be different than (e.g., greater, or less than) the angle A1. In some embodiments, the angle A1 and the angle A2 may be greater than about 0 degrees and less than about 90 degrees, including all sub-ranges and values therebetween. In an embodiment, the angles A1 and A2 can be approximately 45 degrees, or between about 40 degrees and about 50 degrees. Also as shown in FIG. 5A, the second and third sets of agitation elements 432b, 432c are configured with the loops of each set lying in the same radial plane. In other words, the agitation elements of sets 432b, 432c can be set at angles A3, A4 of approximately 90 degrees from the longitudinal axis of the shaft 431. While not depicted, it can be appreciated that additional or alternative sets of agitation elements can be included that are set at angles greater than about 90 degrees from the longitudinal axis of the shaft 431. In such instances, such agitation elements can be directed toward a distal end of the agitation device 430 while also being directed outward toward a vessel wall.

The length (e.g., distance extending from the shaft 431) and/or width of the loops of the sets of agitation elements 432a, 432b, and 432c can be the same or vary in size when extended or expanded. In some embodiments, the length or extension from the shaft 431 of the loops can be between approximately 2 to approximately 20 mm, including all sub-ranges and values therebetween. In some embodiments, the width of the loops can be between approximately 2 to approximately 20 mm, including all sub-ranges and values therebetween. The widths and/or lengths of the loops can be selected to optimize or increase thrombus removal. For example, small width loops can be used to catch and break apart smaller clots. As another example, the width of the loops may be larger to allow for smaller clot particles to pass through after a larger clot has been broken down.

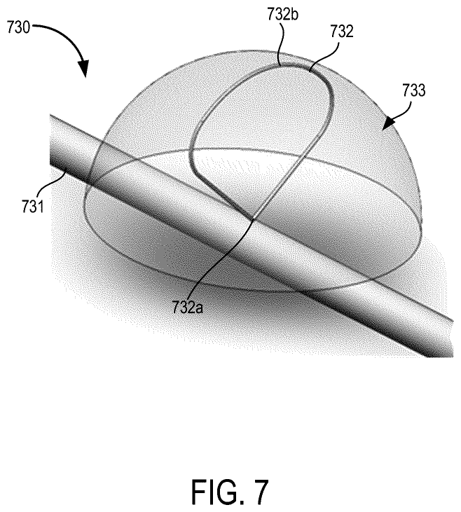

The number and configuration of the sets of agitation elements 432a, 432b, and 432c can be selected to optimize or increase thrombus engagement, reshaping, fragmentation, and/or removal. For example, proximally angled agitation elements, such as the first set of agitation element 432a, can be configured to reach thrombus material that is located at or near a proximal encapsulation element (e.g., encapsulation element 224), while distally angled agitation elements can be configured to reach thrombus material that is located at or near a distal encapsulation element (e.g., encapsulation element 214). In other words, the agitation elements are angled relative to the axis of the catheter system (or specifically, the agitator) to remove thrombus at the periphery of encapsulation elements. Further details of the positioning and/or orientation of the agitation elements are described with reference to FIG. 7 below.

The infusion ports 402 can be located along a distal portion of the shaft 431. In some embodiments, the infusion ports 402 can be arranged in an alternating or offset pattern along the distal portion of the shaft 431, e.g., to distribute fluid within the treatment area. The geometry (e.g., diameter, shape, etc.) of the infusion ports 402 may be configured specifically to allow for a desirable amount of fluid to be delivered with desirable flow characteristics via the shaft 431 with a predetermined period of time. In other words, the size of the infusion ports 402 can be configured to allow for a predetermined rate of fluid delivery. In some embodiments, the infusion ports 402 may include a mesh or filter to prevent clot particles from entering the infusion ports 402. While FIG. 4 depicts a specific arrangement of infusion ports 402, it can be appreciated that the distribution of the infusion ports 402 can be different than that depicted in FIG. 4. For example, the infusion ports may be arranged in a checkboard pattern, a straight line, axial rows, circumferentially, periodically, or any other pattern. In some embodiments, the infusion ports 402 may also be irregularly positioned along the shaft 431 and/or have irregular shapes.

FIG. 5B depicts a front view of the agitation device 430 of FIG. 4. As shown, the sets of agitation elements 432a, 432b, and 432c together can extend substantially around a circumference of the shaft 431. In particular, the sets of agitation elements 432a, 432b, and 432c form a four-leaf clover pattern. In some embodiments, the agitation elements may be arranged in a different, organized pattern. Alternatively, the sets of agitation elements 432a, 432b, and 432c may be arranged with forming a pattern (e.g., randomly, pseudo-randomly, etc.). In use, one or more sets of agitation elements 432a, 432b, and 432c can be configured to contact and deform to the shape of a vessel lumen. As such, when disposed within a vessel, the sets of agitation elements 432a, 432b, and 432c may not form an organized pattern but may deform to the general shape of the vessel. The spacing, angling, and other configurations of the agitation elements can be selected such that, in use, the agitation elements can effectively engage with all or a substantial majority of the thrombus or clot, or selectively engage with portions of the thrombus or clot, within the treatment area.

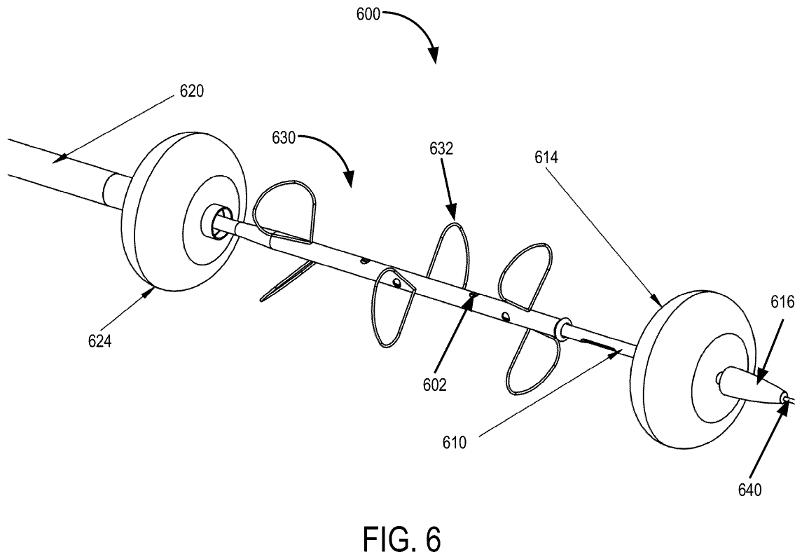

FIG. 6 depicts a perspective view of a catheter system 600 including an agitation device 630 for treating thrombosis, according to embodiments. The catheter system 600 can be functionally and/or structurally similar to other catheter systems described herein (e.g., the catheter systems depicted in FIGS. 1 and 2). For example, the catheter system 600 includes a catheter 610 (e.g., functionally and/or structurally similar to the catheter 110 or 210), a sheath 620 (e.g., functionally and/or structurally similar to the sheath 120 or 220), an agitation device 630 (e.g., functionally and/or structurally similar to the agitation device 130, 230, 330, or 430), and a guidewire 640. As such, certain details of these components are not repeated herein. The catheter system 600 is configured to isolate a treatment area including a thrombus and to reshape, fragment, or break apart the thrombus.

The catheter 610 includes a first encapsulation element 614 (e.g., functionally and/or structurally similar to the first encapsulation element 214 of FIG. 2) and an atraumatic tip 616. The catheter 610 can be configured to be advanced along a guidewire. The first encapsulation element 614 can expand or inflate (e.g., deploy) to contact a vessel wall to define a first end of a treatment area containing a thrombus. The first encapsulation element 614 may be deflated (e.g., undeployed) during advancement of the catheter to the thrombus site. The first encapsulation element 614 can be advanced distal to the thrombus and inflated (e.g., deployed). Once inflated, the first encapsulation element 614 can define one end of the treatment area. The sheath 620 includes a second encapsulation element 624. The second encapsulation element 614 may be deflated (e.g., undeployed) during advancement of the sheath 620 to the thrombus site. The second encapsulation element 624 can be positioned proximal to the thrombus, before, during, or after the first encapsulation element 614 is disposed distal to the thrombus. The second encapsulation element 624 can then be inflated (e.g., deployed). Once inflated, the second encapsulation element 624 can define an opposite end of the treatment area. The first and second encapsulation elements 614, 624 can be inflated (e.g., deployed) in either order or simultaneously and can collectively isolate the treatment area containing the thrombus, e.g., for subsequent reshaping and/or fragmentation of the thrombus and removal of the thrombus. In some embodiments, the second encapsulation element 624 can be inflated before the first encapsulation element 614. Depending on the size and/or distribution of the thrombus, the length of the treatment area can be adjusted, e.g., moving the catheter 610 relative to the sheath 620 to thereby increase or decrease the distance between the first and second encapsulation elements 614, 624. For example, the length of the treatment area can be increased by extending the catheter 610 distally away from the sheath 620, and the length of the treatment area can be reduced by sliding the catheter 610 toward the sheath 620. Depending on the design of the encapsulation elements, the entire system 600 can be moved or repositioned to an additional or adjacent treatment zone in the vasculature with or without deflation (e.g., undeployment) of one or both of the encapsulation elements 614, 624. Once the system 600 has been repositioned in the vasculature, the encapsulation elements 614, 624 can be redeployed, if necessary, to establish another treatment zone containing a thrombus.

The agitation device 630 can be disposed such that its agitation elements 632 are disposed between the first and second encapsulation elements 614, 624. In some embodiments, the agitation device 630 can be advanced along with the catheter 610 and the outer sheath 620 into the vasculature and to the thrombus site. Alternatively, the agitation device 630 can be advanced to the treatment area after the catheter 610 and the outer sheath 620 have been positioned around the treatment area and the first and second encapsulation elements 614, 624 have been inflated or deployed. The agitation elements 632 can be functionally and/or structurally similar to other agitation elements described herein (e.g., agitation elements 332 or 432). The agitation device 630 can also include one or more infusions ports 602 (e.g., functionally and/or structurally similar to the infusion ports 202, 302, or 402). The agitation elements 632 can be configured to mechanically reshape or break up a clot within the treatment area. The infusion ports 602 are configured to deliver fluid to the treatment area, e.g., for softening or lysing the clot, for delivering a therapeutic agent, or for other delivery of fluid (e.g., saline, vascular contrast agent).

The catheter 610, the outer sheath 620, and the agitation device 630 can be concentrically arranged, e.g., with the catheter being disposed within a lumen of the agitation device, and the agitation device and the catheter being disposed within a lumen of the outer sheath. The catheter 610 an include or define a lumen that can receive a guidewire 640, as depicted in FIG. 6 As such, the entire catheter system 600 can be advanced over the guidewire 640 to a thrombus site.

In use, the guidewire 640 can be inserted into the vessel and advanced to a thrombus site. The catheter system 600 can then be advanced over the guidewire 640 to the thrombus site, and the encapsulation elements 614 and 624 can be deployed to isolate the treatment area or region around the thrombus site. The agitation elements 632 of the agitator 630 can then be deployed, e.g., by extending out the agitation device 630. And the agitation elements 632 can be configured to reshape, fragment, or break apart the thrombus such that the thrombus can be removed from the vessel. In some embodiments, a suction can be applied to the treatment area, e.g., via a central lumen of the outer sheath 620 or a separate catheter lumen, to aspirate the thrombus (once reshaped or fragmented) out of the treatment area. In some embodiments, a thrombolytic agent can be delivered to the treatment area via infusion ports 602 before using the agitation elements 632 to reshape the thrombus. Alternatively or additionally, an infusion fluid such as filtered blood or saline can be delivered to the treatment area during or after removal of the thrombus.

FIG. 7 schematically depicts a region 733 that defines the possible positions or orientations of an agitation element 732 (e.g., functionally and/or structurally similar to the agitation element 332a/332b of FIG. 3) of an agitation device 730 (e.g., functionally and/or structurally similar to the agitation device 130 of FIG. 1), according to embodiments. As described above with reference to FIGS. 4-5B, an agitation element of an agitation device can be orientated and/or positioned in one of a plurality of possible orientations or positions. In the case where the agitation element is a loop that is fixedly coupled at a point 732a to a shaft, the agitation element can be set to any one of the positions or orientations that fall within the region 733 around that fixed point. In particular, the region 733 represents the hemispherical area, defined by the attachment position 732a to the shaft and the point 732b on the agitator element furthest from the attachment position, in which the agitation element 732 can be oriented or positioned, when mounted at a fixed point on the shaft 731. The orientation of the agitation element 732 within the region 733 can be selected for a specific application of the agitation device 730 or may be configured to meet a wide range of applications.

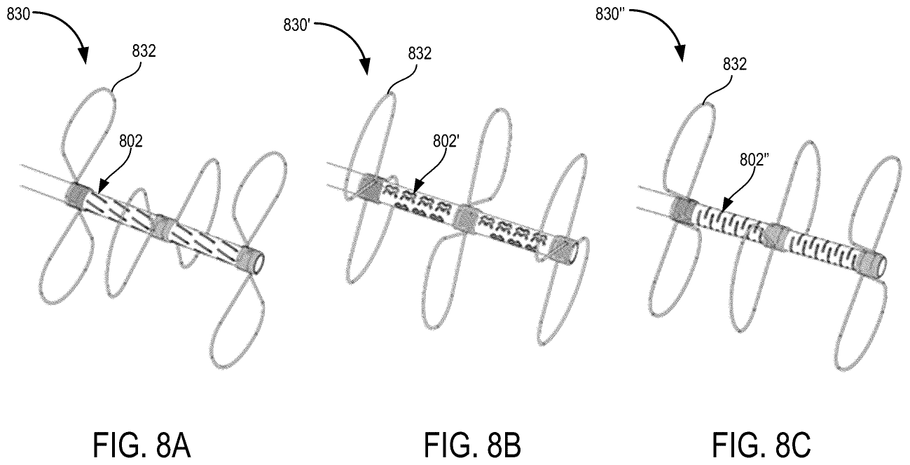

FIGS. 8A-8C schematically depict different infusion segments including fenestrations 802, 802′, and 802″ of various agitation devices 830, 830′, and 830″ for treating thrombosis, according to embodiments. The fenestrations 802, 802′, and 802″ can be arranged in different patterns, as shown in FIGS. 8A-8C. While FIGS. 8A-8C depict three example fenestration patterns, it can be appreciated that additional fenestration patterns can be used to achieve desired results (e.g., amount, volume, rate, or distribution of fluid infused into the treatment area). For example, additional fenestration patterns may be used depending on the flow pattern, flow distribution, flow rate, fluid dynamics, infusion speed, etc. desired in a particular application.

In some embodiments, the fenestrations 802, 802′, and 802″ can function as infusion ports, e.g., similar to other infusion ports described herein (e.g., infusion ports 202, 302, or 402). The configuration or arrangement (e.g., size, shape, number, pattern, etc.) of the infusion ports or fenestrations can be configured to deliver specific amounts of fluid to the treatment area over a predetermined period of time. In some embodiments, the configuration or arrangement of the fenestrations may be constant throughout the infusion segment. In some embodiments, the configuration or arrangement of the fenestrations may vary throughout the infusion segment. In some embodiments, the fenestrations 802, 802′, and 802″ can be used to control the response of the agitator shaft to various loading conditions, including, for example, torsional and axial loading or any combination thereof. In some embodiments, the fenestrations may be present on up to 100% of the surface of the agitation device in the infusion segment, e.g., to provide for control of the response of the agitator shaft to various loading conditions.

FIG. 8A depicts an agitation device 830 having a first set of fenestrations 802 arranged according to a first pattern. The first fenestration pattern is located on the agitation device 830 between adjacent agitation elements 832. The first fenestration pattern includes axial slit-shaped openings or ports that are arranged circumferentially in repeating groups. The openings are slightly angled with respect to the longitudinal direction of the shaft of the agitation device 830. In some embodiments, the shape, angle, and distribution of the infusion ports of this first fenestration pattern can be selected to achieve specific infusion conditions and/or to control the agitator shafts response to various loading conditions. The first fenestration pattern can be easily manufactured given the straight or slotted configuration of the fenestrations.

FIG. 8B depicts an agitation device 830′ having a second set of fenestrations 802′ arranged according to a second pattern. The second fenestration pattern is located on the agitation device 830 between adjacent agitation elements 832. The second fenestration pattern includes axially undulating inclusion openings or ports that are arranged circumferentially along the agitation device 830′. In some embodiments, the openings or ports may be formed in pairs, but in other embodiments, the openings or ports may be formed in larger or smaller groups. In some embodiments, the undulating pattern can repeat. In some embodiments, the shape, angle, and distribution of the infusion ports of this second fenestration pattern can be selected to achieve specific infusion conditions and/or to control the agitator shafts response to various loading conditions.

FIG. 8C depicts an agitation device 830″ having a third set of fenestrations 802″ arranged according to a third fenestration pattern. The third fenestration pattern is located on the agitation device 830″ between adjacent agitation elements 832. The third fenestration pattern includes circumferential infusion ports or openings that are arranged in an axially alternating pattern along the agitation device 830″. In some embodiments, the shape, angle, and distribution of the infusion ports of this third fenestration pattern can be selected to achieve specific infusion conditions and/or to control the agitator shafts response to various loading conditions. For example, the third fenestration pattern may provide more flexibility than the first fenestration pattern 802 and the second fenestration patter 802′.

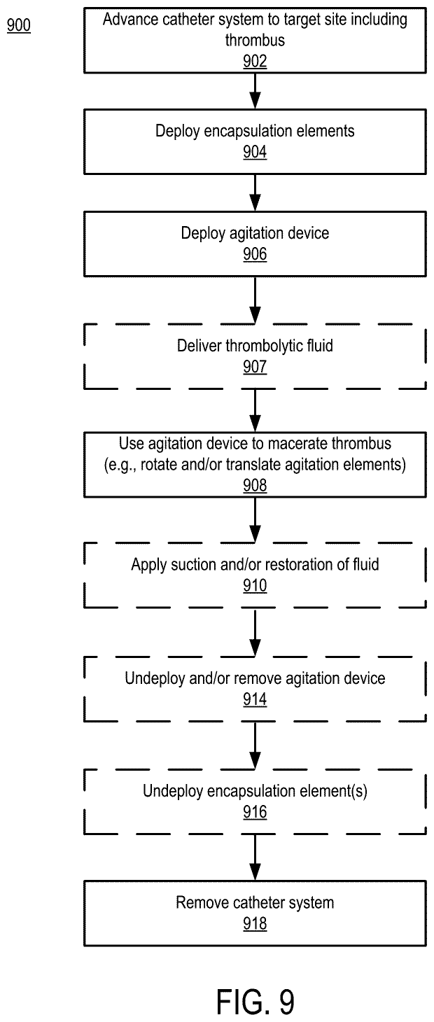

FIG. 9 is a flow diagram of a method 900 of performing a thrombectomy procedure using any of the catheter systems or components thereof described herein, according to embodiments. The method 900 can be facilitated by a medical professional.

At 902, a catheter system (e.g., catheter system 100 or any of the other catheter systems described herein) is advanced to a target site including a thrombus. The catheter system may be advanced along a guidewire (e.g., guidewire 640) that was inserted first into a vessel.

During insertion, the encapsulation elements (e.g., first encapsulation element 213 and second encapsulation element 224 and/or other encapsulation elements described herein) can be deflated or undeployed to allow the catheter system to translate through the vessel and to reach the target area. At 904, once the target area is reached, the encapsulation elements may be deployed (e.g., filled, inflated, actuated, etc.) to isolate a treatment area that contains the thrombus. Deploying the encapsulation elements may include inflating the encapsulation elements using an inflation source (e.g., inflation source 170). In some embodiments, the encapsulation elements can be inflated until they contact the vessel wall and seal off the treatment area. In some embodiments, the encapsulation elements may be filled to predetermine parameters (e.g., a pressure threshold, volume, size, etc.).

At 906, an agitation device (e.g., agitation device 130 or other agitation devices described herein) is deployed. Deploying the agitation device may include advancing or extending a distal end of the agitation device into the treatment area. As described above with respect to the agitation devices disclosed herein, the agitation device can include one or more agitation elements which can be configured to self-expand or be deployed within the treatment area.

Optionally, at 907, the catheter system delivers thrombolytic fluid into the treatment area. The thrombolytic fluid may be delivered by the infusion source via the infusion ports. The thrombolytic fluid may aid in breaking apart and dissociation of the thrombus before, during, or after maceration. Infusion may be continuous, periodic, or may be manually controlled by a medical professional. In some embodiments, the thrombolytic fluid can be delivered to the treatment area before using the agitation device to reshape, soften, or dissolve the thrombus. Alternatively, the thrombolytic fluid can be delivered during or after using the agitation device to aid in further breaking down thrombus prior to removal.

At 908, the agitation device is used to reshape, macerate, or otherwise engage with the thrombus. Reshaping or macerating the thrombus may include rotating and/or translating the agitation elements within the treatment area to reshape or break up the thrombus into smaller particles. In some embodiments, multiple phases of clot removal may be employed. For example, a first phase can include detaching the thrombus from the vessel wall, and a second phase may include reshaping or breaking up the thrombus.

Optionally, at 910, the catheter system applies suction to the treatment area and/or restores fluid within the treatment area. The catheter system may apply suction via a suction source to remove particles of the thrombus from the treatment area. Removing particles may also remove fluid within the treatment area. In some embodiments, the removed fluid may be restored (e.g., replaced, replenished, etc.) to prevent a vacuum from forming within the vessel and potentially collapsing the vessel. The fluid may be replaced via a separate infusion source (e.g., infusion source 160 or other infusion sources described herein). In some embodiments, the catheter system does not apply suction. In such cases, once the thrombus has been detached from the vessel wall, the catheter system can be removed and the thrombus along with it.

Optionally, at 914, the agitation device is undeployed and/or removed after the thrombus is reshaped or broken apart. Undeploying the agitation device may include retracting the agitation device into an outer sheath (e.g., outer sheath 120 and/or other outer sheaths described herein). In some embodiments, the agitation device may be completely removed from the treatment area or the catheter device. In some embodiments, the agitation device may not be removed and may be left in the treatment area. At 916, one or more of the encapsulation elements may be undeployed before removing the catheter system from the patient. For example, a proximal encapsulation element (e.g., encapsulation element 224 or other encapsulation elements described herein) can be undeployed while a distal encapsulation element (e.g., encapsulation element 214 or other encapsulation elements described herein) can remained deployed or vice versa. By keeping at least one encapsulation element deployed, it can prevent smaller particles of the thrombus that may still be within the treatment region from re-entering the vasculature (e.g., embolization). Undeploying an encapsulation element may allow for easier removal of the catheter device through the vessel. In some embodiments, both encapsulation elements may be undeployed prior to removal.

At 918, the catheter system is removed, e.g., by retracting the catheter system out of the patient vasculature.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The specific examples and descriptions herein are exemplary in nature and embodiments may be developed by those skilled in the art based on the material taught herein without departing from the scope of the present invention.

1. An apparatus, comprising:
a shaft defining a lumen, the shaft including a distal portion configured to be disposed within a vessel lumen near a thrombus;
a plurality of fenestrations disposed on the shaft, the plurality of fenestrations being fluidically coupled to the lumen, the lumen and the plurality of fenestrations configured to deliver a fluid into a treatment area containing the thrombus;
at least one set of one or more agitation elements disposed at different locations along the distal portion of the shaft, each set of agitation elements including at least one agitation element configured to extend from an outer surface of the shaft to a wall of the vessel lumen, the sets of agitation elements configured to independently translate and rotate to reshape or fragment the thrombus.

2. The apparatus of claim 1, wherein each set of agitation elements includes a plurality of loops disposed circumferentially around the shaft.

3. The apparatus of claim 2, wherein each of the plurality of loops is configured to contact the wall of the vessel lumen and to deform to a shape of the vessel lumen.

4. The apparatus of claim 2, wherein each of the plurality of loops is configured to have a length of between about 2 mm and about 20 mm.

5. The apparatus of claim 2, wherein each of the plurality of loops is configured to have a width of between about 2 mm and about 20 mm.

6. The apparatus of claim 2, wherein the sets of agitation elements include a first set of agitation elements having a first plurality of loops and a second set of agitation elements having a second plurality of loops,
the first plurality of loops being set at an angle relative to a longitudinal axis of the shaft that is different than an angle at which the second plurality of loops is set relative to the longitudinal axis of the shaft.

7. (canceled)

8. The apparatus of claim 2, wherein the sets of agitation elements include a first set of agitation elements having a first plurality of loops and a second set of agitation elements having a second plurality of loops,
the first plurality of loops being offset circumferentially from the second set of loops.

9. The apparatus of claim 8, wherein the first plurality of loops is offset from the second plurality of loop by about 90 degrees or less.

10. The apparatus of claim 2, wherein the plurality of loops is formed of at least one of: PEEK, Pebax, or nitinol.

11. The apparatus of claim 1, wherein the plurality of fenestrations is disposed in a pattern along a length of the distal portion of the shaft.

12. The apparatus of claim 1, wherein the plurality of fenestrations is configured to deliver the fluid into the treatment area at a predefined rate or to deliver a predefined volume of the fluid over a set period of time into the treatment area.

13. The apparatus of claim 1, wherein the fluid includes at least one of: a thrombolytic fluid, a therapeutic fluid, a diagnostic fluid, or saline.

14. The apparatus of claim 1, wherein the shaft is configured to be advanced to the treatment site over another medical device.

15. The apparatus of claim 1, wherein the shaft is configured to be translated or rotated to translate or rotate the sets of agitation elements.

16. The apparatus of claim 1, wherein each of the sets of agitation elements includes at least two agitation elements positioned at different locations about a circumference of the shaft.

17. An apparatus, comprising:
a catheter defining a first lumen and including a first encapsulation element disposed on a distal end of the catheter, the first lumen configured to receive a guidewire to enable the catheter to be advanced along the guidewire to a treatment area in a vessel lumen that includes a thrombus;
an outer sheath defining a second lumen and including a second encapsulation element disposed on a distal end of the outer sheath, the first and second encapsulation elements each configured to transition from an undeployed state to a deployed state, the first and second encapsulation elements in the deployed state collectively configured to isolate the treatment area including the thrombus;
an agitation device disposable within the second lumen and defining a third lumen configured to receive the catheter such that the agitation element can be advanced along the catheter, the agitation element including a plurality of agitation elements, the plurality of agitation elements configured to be disposed between the first and second encapsulation elements such that the plurality of agitation elements can engage with the thrombus and reshape or fragment the thrombus; and
a plurality of infusion ports disposed on at least one of the catheter, the outer sheath, or the agitation device, the plurality of infusion ports being fluidically coupled to at least one of the first, second, or third lumens such that a fluid can be delivered via the first, second, or third lumen and the plurality of infusion ports into the treatment area.

18. (canceled)

19. (canceled)

20. (canceled)

21. (canceled)

22. (canceled)

23. (canceled)

24. The apparatus of claim 17, further comprising an extracorporeal assembly, the extracorporeal assembly including a connector configured to couple the first and second encapsulation elements to an inflation source.

25. The apparatus of claim 17, further comprising an extracorporeal assembly, the extracorporeal assembly including a connector configured to couple the plurality of fenestrations to an infusion source.

26. The apparatus of claim 17, further comprising an extracorporeal assembly, the extracorporeal assembly including a connector configured to couple the second lumen of the outer sheath to a vacuum source such that particles of thrombus within the treatment area can be aspirated out of the treatment area via the second lumen by applying the vacuum.

27. (canceled)

28. (canceled)

29. A method, comprising:
deploying first and second encapsulation elements of a catheter system disposed within a vessel lumen to isolate a treatment area of the vessel lumen containing a thrombus;
deploying a plurality of agitation elements of the catheter system within the treatment area, the plurality of agitation elements when deployed being configured to contact a wall of the vessel lumen; and
translating and rotating the plurality of agitation elements to reshape or fragment the thrombus.

30. (canceled)

31. (canceled)

32. (canceled)

33. (canceled)
